(19) United States
(12) Patent Application Publication
(10) Pub. No.: US 2014/0162266 A1
(43) Pub. Date: Jun. 12, 2014

(54) METHODS FOR POLYMERASE CHAIN REACTION COPY NUMBER VARIATION ASSAYS

(71) Applicant: Bio-Rad Laboratories, Inc., Hercules, CA (US)

(72) Inventors: Niels Klitgord, Kensington, CA (US); John Regan, San Mateo, CA (US); Svilen Tzonev, Pleasanton, CA (US); Eli Hefner, Hercules, CA (US); Jennifer R. Berman, San Carlos, CA (US)

(73) Assignee: Bio-Rad Laboratories, Inc., Hercules, CA (US)

(21) Appl. No.: 14/097,923

(22) Filed: Dec. 5, 2013

Related U.S. Application Data
(60) Provisional application No. 61/733,881, filed on Dec. 5, 2012.

(51) Int. Cl. C12Q 1/68

(52) U.S. Cl. CPC C12Q 1/6844
USPC 435/6.11

(57) ABSTRACT

This disclosure provides methods for measuring the copy number for highly amplified and/or abundant genomic loci. Recognized herein is a need for methods for determining nucleic acid copy number, particularly in instances where one locus to be quantified (i.e., the target) is relatively more abundant than a locus of known abundance (i.e., the reference). In some cases, the method involves combining a query nucleic acid sample with a diluting nucleic acid sample and measuring the relative copy number of a target sequence compared with a reference sequence in the combined sample.

### CROSS-REFERENCE

This application claims the benefit of U.S. Provisional Patent Application Ser. No. 61/733,881, filed Dec. 5, 2012, which is incorporated herein by reference in its entirety.

### BACKGROUND

An assay can be an investigative procedure for determining, among other things, the presence, quantity, activity, and/or other properties or characteristics of components in a sample. Sometimes, the components of interest within a sample (e.g., a nucleic acid, an enzyme, a virus, a bacterium) are only minor constituents of the sample and may, therefore, be difficult to detect or quantify.

An example of a biological assay is a polymerase chain reaction (PCR) assay. Certain types of PCR can be quantitative in specific settings. For example, real-time PCR (which can involve monitoring the progression of amplification using fluorescence probes) can permit quantification of target nucleic acids in a sample, particularly where the target nucleic acids are somewhat abundant.

### SUMMARY

Recognized herein is the need for methods for determining nucleic acid copy number, particularly in instances where one locus to be quantified (i.e., the target) is relatively more abundant than a locus of known abundance (i.e., the reference). Provided herein are methods for measuring the copy number for highly amplified and/or abundant genomic loci.

An aspect of the present disclosure provides a method for determining nucleic acid copy number, the method comprising: (a) providing a query nucleic acid sample comprising a target sequence and a reference sequence; (b) combining with the query nucleic acid sample, a diluting nucleic acid sample having a known copy number of the reference sequence; and (c) measuring the relative copy number of the target sequence compared to the reference sequence in the combined sample using digital polymerase chain reaction (PCR). The target sequence can be a nucleic acid sequence. The reference sequence can be a nucleic acid sequence.

In some embodiments, nucleic acid molecules in the query nucleic acid sample comprise at least one copy of the reference sequence and at least one copy of the target sequence on the same nucleic acid molecule.

In some embodiments, the digital PCR is performed in an emulsion having droplets.

In some embodiments, the droplets have zero or one copy of the target sequence and/or reference sequence.

In some embodiments, the quantity of query nucleic acid sample is measured or known to within an accuracy of 5%.

In some embodiments, there are at least 100-fold more copies of the target sequence than the reference sequence in the query nucleic acid sample.

In some embodiments, there are at least 10,000-fold more copies of the target sequence than the reference sequence in the query nucleic acid sample.

In some embodiments, a quantity of the diluting nucleic acid sample is combined with a quantity of the query nucleic acid sample such that the ratio of the copy number of the target sequence to the copy number of the reference sequence is between about 0.1 and 10.

In some embodiments, a quantity of the diluting nucleic acid sample is combined with a quantity of the query nucleic acid sample such that the copy number of the target sequence and the copy number of the reference sequence are within 20% of the minimal Poisson statistical uncertainty.

In some embodiments, the target sequence is a deoxyribonucleic acid sequence.

In some embodiments, the reference sequence is a deoxyribonucleic acid sequence.

In some embodiments, the copy number of the target sequence in the query nucleic acid sample is determined within an accuracy of 5%.

An aspect of the present disclosure provides a method for performing digital polymerase chain reaction (PCR), the method comprising: (a) providing a query nucleic acid sample comprising a target sequence and a first reference sequence; (b) combining with the query nucleic acid sample, a diluting nucleic acid sample having the first reference sequence and a second reference sequence; (c) dividing the combined sample into a plurality of reaction volumes; (d) performing a PCR reaction on the reaction volumes to determine the concentration of the target sequence, the first reference sequence and the second reference sequence in the combined sample; and (e) calculating the concentration of the target sequence in the query nucleic acid sample.

In some embodiments, the reaction volumes are droplets of an emulsion.

In some embodiments, the copy number of the first reference sequence in the diluting nucleic acid sample is known.

In some embodiments, the copy number of the second reference sequence in the diluting nucleic acid sample is known.

In some embodiments, the PCR reaction in (d) comprises pairs of primers that anneal to portions of the target sequence, the first reference sequence, and/or the second reference sequence.

In some embodiments, the calculation in (e) is performed by a computer.

In some embodiments, there are at least 100-fold more copies of the target sequence than the first reference sequence in the query nucleic acid sample.

In some embodiments, a quantity of the diluting nucleic acid sample is combined with a quantity of the query nucleic acid sample such that the ratio of the copy number of the target sequence to the copy number of the first reference sequence is between about 0.1 and 10.

In some embodiments, a quantity of the diluting nucleic acid sample is combined with a quantity of the query nucleic acid sample such that the copy number of the target sequence and the copy number of the first reference sequence are within 20% of the minimal Poisson statistical uncertainty.

In some embodiments, the target sequence is a deoxyribonucleic acid sequence.

In some embodiments, the reference sequence is a deoxyribonucleic acid sequence.

In some embodiments, the copy number of the target sequence in the query nucleic acid sample is determined within an accuracy of 5%.

In some embodiments, the second reference sequence is not found on the query nucleic acid sample.

Another aspect of the present disclosure provides a computer readable medium comprising machine executable code which, upon execution by one or more computer processors, implements any of the methods above or elsewhere herein.

Another aspect of the present disclosure provides a system comprising one or more computer processors and memory comprising machine executable code which, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also referred to as “Figures” or “FIGs.”) of which:

FIG. 1A and FIG. 1B schematically illustrate a droplet generation system of the present disclosure;

FIG. 2 schematically illustrates a droplet detection system of the present disclosure;

FIG. 3 shows a computer system that can be used to implement methods of the present disclosure;

FIG. 4 shows an example method for determining nucleic acid copy number for highly amplified and/or highly expressed loci;

FIG. 5 shows an example of a two channel detector showing locations of the clusters;

FIG. 6 shows an example of a triplex assay; and

FIG. 7 shows an example of a determination of copy number for the Myc gene.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed in practicing the invention.

The tern “nucleic acid,” as used herein, generally refers to a molecule comprising one or more of the nucleic acid bases adenine (A), cytosine (C), thymine (T), guanine (G), uracil (U), or a derivative thereof. Nucleic acids include deoxyribonucleic acid (DNA), ribonucleic acid (RNA), as well as any derivatives or modifications thereof. Nucleic acids can be polymeric molecules having a sequence comprising the bases adenine (A), cytosine (C), thymine (T), guanine (G), uracil (U), and derivatives thereof. In some instances, the nucleic acid is a peptide nucleic acid (PNA).

The term “polymerase chain reaction” (PCR), as used herein, generally refers to a biochemical technology used in molecular biology to amplify a piece of DNA (i.e., a target). The amplification can be across several orders of magnitude, sometimes starting from a single or a few copies of the target and generating thousands to millions of copies of a particular DNA sequence.

PCR can use thermal cycling, comprising cycles of repeated heating and cooling of the reaction for DNA melting and enzymatic replication of the DNA. These thermal cycling operations can physically separate the two strands in a DNA double helix at a high temperature in a process called DNA melting. At a lower temperature, each strand can then be used as the template in DNA synthesis by the DNA polymerase to selectively amplify the target DNA. The selectivity of PCR can result from the use of primers (short DNA fragments) that are complementary to the DNA region targeted for amplification under specific thermal cycling conditions.

Primers containing sequences complementary to the target region along with a DNA polymerase can be the principal components to achieve selective and repeated amplification. As PCR progresses, the DNA generated can be used as a template for replication, setting in motion a chain reaction in which the DNA template is exponentially amplified. PCR can be extensively modified to perform a wide array of genetic manipulations.

PCR applications can employ a heat-stable DNA polymerase, such as Taq polymerase, an enzyme originally isolated from the bacterium Thermus aquaticus. This DNA polymerase can enzymatically assemble a new DNA strand from the nucleotides, e.g., by using single-stranded DNA as a template and DNA oligonucleotides (also called DNA primers) for initiation of DNA synthesis.

The term “channel,” as used herein, generally refers to a flow path for conveying a fluid from one point to another. A fluid can be, for example, a gas, a liquid, a mixture of liquids, or a solid-liquid mixture.

The term “downstream” and “upstream,” as used herein, generally refer to the position of a species, such as a droplet, along a system or device(s), such as along a fluid flow path in a droplet generator. A first droplet downstream of a second droplet can be further along a fluid flow path than the second droplet, either in the same device or a separate device. The devices may or may not be connected, such as by a flow path. The second droplet in such a case is upstream of the first droplet.

The term “emulsion,” as used herein, generally refers to a mixture of two or more fluids that are normally immiscible. An emulsion can include a first phase in a second phase, such as an aqueous phase in an oil phase. In some cases, an emulsion includes more than two phases. It may also include multiple emulsions. Moreover, in some examples, an emulsion may include particulates that may function to stabilize the emulsion and/or function as a coating (e.g., gel-like coating), such as a droplet skin.

Digital PCR Copy Number Variation Assays

The present disclosure provides methods for detecting nucleic acid copy number and calculating copy number variation (CNV) in amplification assays, such as PCR assays. Such methods may be employed using devices and systems provided herein, such as emulsion (droplet) based systems.

Digital PCR Copy Number Variation (CNV) assays can assess the relative genomic abundance of one locus (the target) relative to the abundance of known copy number region (reference). Accurately determining the correct copy number for highly amplified loci can be challenging since the DNA load required for accurate target quantification may be much smaller than needed for accurate reference quantification. As a result, when measuring copy number using a duplex assay, a user may be forced to load an amount of deoxyribonucleic acid (DNA) that may be suboptimal for the highest accuracy target and reference quantification. In some cases, the greater the degree of target amplification, the greater this problem becomes, as the uncertainty in digital measurements increases towards to the extreme ends of the instrument's dynamic range.

This disclosure provides methods for performing nucleic acid CNV assays, such as PCR (e.g., digital PCR and/or real-time PCR) CNV assays. Assays may be performed on various nucleic acids, such as deoxyribonucleic acid (DNA), ribonucleic acid (RNA), or variants thereof.

Real-time polymerase chain reaction (RT-PCR) is a laboratory technique based on PCR, which can be used to amplify and simultaneously quantify a targeted DNA molecule. Real-time PCR can be combined with reverse transcription to quantify messenger RNA and non-coding RNA in cells or tissues.

For one or more specific sequences in a DNA sample, real time-PCR can allow both detection and quantification. The quantity can be either an absolute number of copies or a relative amount when normalized to DNA input or additional normalizing genes (i.e., reference).

The procedure can follow the principle of polymerase chain reaction with the feature that the amplified DNA is detected as the reaction progresses in real time. Two common methods for detection of products in real-time PCR (RT-PCR) include: (1) non-specific fluorescent dyes that intercalate with any double-stranded DNA, and (2) sequence-specific DNA probes comprising oligonucleotides that are labeled with a fluorescent reporter which permits detection only after hybridization of the probe with its complementary DNA target.

In RT-PCR methods using double stranded DNA-binding dyes as reporters, a DNA-binding dye binds to double-stranded (ds) DNA in PCR, causing fluorescence of the dye. An increase in DNA product during PCR therefore leads to an increase in fluorescence intensity and is measured at each cycle, thus allowing DNA concentrations to be quantified. However, dsDNA dyes such as SYBR Green can bind to all dsDNA PCR products, including nonspecific PCR products (such as primer dimer). This can potentially interfere with, or prevent, accurate quantification of the intended target sequence.

Method of the present disclosure can comprise preparing a PCR reaction, with the addition of fluorescent dsDNA dye. The reaction can be run in a real-time PCR instrument, and after each cycle, the levels of fluorescence can be measured with a detector. In some cases, the dye only fluoresces when bound to the dsDNA (i.e., the PCR product). The dsDNA concentration in the PCR can be determined with reference to a standard dilution (i.e., calibration).

The values obtained do not have absolute units associated with them in some cases (i.e., mRNA copies/cell). A comparison of a measured DNA/RNA sample to a standard dilution can give a fraction or ratio of the sample relative to the reference standard, allowing only relative comparisons between different samples or experimental conditions. To improve accuracy in the quantification, one can normalize expression of a target gene to a stably expressed gene and/or normalize a target loci to a loci of known copy number. This normalization can correct possible differences in RNA quantity or quality across experimental samples.

Some RT-PCR methods use a fluorescent reporter probe. Fluorescent reporter probes can detect the DNA containing the probe sequence. Use of the reporter probe can significantly increase specificity compared with double stranded DNA binding dyes, and allow for quantification even in the presence of non-specific DNA amplification. Fluorescent probes can be used in multiplex assays (i.e., for detection of several genes in the same reaction) based on specific probes with different-colored labels, provided that all targeted genes are amplified with similar efficiency. The specificity of fluorescent reporter probes can also prevent interference of measurements caused by primer dimers, which are potentially undesirable by-products in PCR.

The fluorescent reporter probes method can rely on a DNA-based probe with a fluorescent reporter at one end and a quencher of fluorescence at the opposite end of the probe. The close proximity of the reporter to the quencher can prevent detection of its fluorescence. Breakdown of the probe by the 5′ to 3′ exonuclease activity of the Taq polymerase can break the reporter-quencher proximity and thus allows unquenched emission of fluorescence, which can be detected after excitation with a laser. An increase in the product targeted by the reporter probe at each PCR cycle can therefore cause a proportional increase in fluorescence due to the breakdown of the probe and release of the reporter.

Methods of the present disclosure can comprise preparing a PCR reaction with the reporter probe added. As the reaction commences, during the annealing stage of the PCR both probe and primers can anneal to the DNA target. Polymerization of a new DNA strand can be initiated from the primers, and once the polymerase reaches the probe, its 5′-3′-exonuclease can degrade the probe, physically separating the fluorescent reporter from the quencher, resulting in an increase in fluorescence. Fluorescence can be detected and measured in a real-time PCR machine, and its geometric increase corresponding to exponential increase of the product is used to determine the quantification cycle (C_{q}) in each reaction.

Digital Polymerase Chain Reaction (digital PCR or dPCR) is a variation of conventional polymerase chain reaction methods that can be used to directly quantify and clonally amplify nucleic acids including DNA or RNA. One difference between dPCR and traditional PCR lies in the method of measuring nucleic acids amounts, with dPCR being a more precise method than PCR. PCR carries out one reaction per single sample. dPCR also carries out a single reaction within a sample, however the sample is separated into a large number of partitions (e.g., droplets) and the reaction is carried out in each partition individually. This separation allows a more reliable collection and sensitive measurement of nucleic acid amounts. This approach can be used to study variations and mutations in gene sequences, such as copy number variations and point mutations.

In a dPCR method, a sample can be partitioned so that individual nucleic acid molecules within the sample are localized and concentrated within many separate regions (e.g., at least 100, at least 1000, or at least 10000 regions). The capture or isolation of individual nucleic acid molecules can be performed in micro well plates, capillaries, the dispersed phase of an emulsion, and arrays of miniaturized chambers, as well as on nucleic acid binding surfaces. The partitioning of the sample can allow for the estimation of the number of different molecules by assuming that the molecule population follows the Poisson distribution. As a result, each partition will contain “0” or “1” molecules, or a negative or positive reaction, respectively. After PCR amplification, nucleic acids may be quantified by counting the regions that contain PCR end-product (i.e., “1” or positive reactions). In conventional PCR, the number of PCR amplification cycles is proportional to the starting copy number. However, dPCR is not usually dependent on the number of amplification cycles to determine the initial sample amount, eliminating the reliance on uncertain exponential data to quantify target nucleic acids and therefore provides more absolute quantification.

Methods of the disclosure are based at least in part on the unexpected realization that the uncertainty in copy number measurements and/or digital measurements can be reduced (e.g., leading to more precise and more accurate CNV estimates) by combining a query DNA with a known amount of a diluting DNA (i.e., DNA with a known amounts of target and reference sequence).

In an aspect of the disclosure, a method for determining nucleic acid copy number comprises providing a query nucleic acid sample comprising a reference sequence of known copy number and a target sequence of unknown copy number. Next, the query nucleic acid sample is combined with a diluting nucleic acid sample having a known copy number of the target and reference sequences. The relative copy number of the target sequence, compared to the reference sequence in the combined sample, can then be measured.

Multiple reference sequences can be used. A first reference sequence can be used to establish the amount of diluting DNA added and a second reference sequence can be used to quantify the total number of haploid genome equivalents in the sample. In some cases, a reference assay detects both the queried sample and the diluting sample. Then, a second reference assay detects a sequence unique to the diluting DNA so that the amount of dilution can be quantified.

In an example, a method for performing digital polymerase chain reaction (PCR) comprises providing a query nucleic acid sample comprising a target sequence and a first reference sequence, and combining, with the query nucleic acid sample, a diluting nucleic acid sample having the first reference sequence and a second reference sequence. Next, the combined sample is divided into a plurality of reaction volumes. A PCR reaction is then performed on the reaction volumes to determine the concentration of the target sequence, the first reference sequence and the second reference sequence in the combined sample. The concentration of the target sequence in the query nucleic acid sample is then calculated.

In an example, the dynamic range of the Digital PCR Copy Number Variation assays described here can be about 100-fold. Query nucleic acid samples having at least 100-fold more copies of the target sequence than the reference sequence can be brought into an acceptable dynamic range by adding diluting nucleic acid (e.g., having a known number of copies of the reference sequence). Digital PCR Copy Number Variation assays can then be performed using droplets encapsulating nucleic acid from the combined sample as described here. The amount of diluting nucleic acid that is added can be measured and compensated for with software or computer systems as described here.

The query nucleic acid sample can have 1, 2, 3, 4, 5, 6, 7, 8, 9, 10, 11, 12, 13, 14, 15, 16, 17, 18, 20, 21, 22, 23, 24, 25, 26, 27, 28, 29, 30, or even more copies of the target sequence (e.g., per haploid genome). The query nucleic acid sample can have 1, 2, 3, 4, 5, 6, 7, 8, 9, 10, 11, 12, 13, 14, 15, 16, 17, 18, 20, 21, 22, 23, 24, 25, 26, 27, 28, 29, 30, or even more copies of the reference sequence (e.g., per haploid genome). In some instances, the query nucleic acid sample has about 10, about 50, about 100, about 500, about 1000, about 5000, about 10000 or more copies of the target sequence. In some cases, the query nucleic acid sample has at least about 10, at least about 50, at least about 100, at least about 500, at least about 1000, at least about 5000, at least about 10000 or more copies of the target sequence.

The target nucleic acid can be highly abundant and/or highly amplified in comparison to the reference nucleic acid in the query nucleic acid sample. In some embodiments, there are about 5-fold, about 10-fold, about 50-fold, about 100-fold, about 500-fold, about 1000-fold, about 5000-fold, about 10000-fold, about 50000-fold, about 100000-fold, about 500000-fold, about 1000000-fold, about 5000000-fold, about 10000000-fold, or more copies of the target sequence than the reference sequence in the query nucleic acid sample. In some embodiments, there are at least about 5-fold, at least about 10-fold, at least about 50-fold, at least about 100-fold, at least about 500-fold, at least about 1000-fold, at least about 5000-fold, at least about 10000-fold, at least about 50000-fold, at least about 100000-fold, at least about 500000-fold, at least about 1000000-fold, at least about 5000000-fold, at least about 10000000-fold, or more copies of the target sequence than the reference sequence in the query nucleic acid sample.

In some embodiments, nucleic acid molecules in the query nucleic acid sample comprise at least one copy of the reference sequence and at least one copy of the target sequence on the same nucleic acid molecule (e.g., on a haploid genome). In some instances, the target sequence and the reference sequence are on different molecules (e.g., the query nucleic acid sample includes mRNA of a highly expressed target sequence and mRNA of a less highly expressed reference sequence (e.g., the target sequence is at least 10-fold, at least 100-fold or at least 1,000-fold more abundant)).

The diluting nucleic acid sample may have 1, 2, 3, 4, 5, 6, 7, 8, 9, 10, 11, 12, 13, 14, 15, 16, 17, 18, 20, 21, 22, 23, 24, 25, 26, 27, 28, 29, 30, or even more copies of the reference sequence (e.g., per haploid genome). In some cases, the diluting nucleic acid sample has 1 copy/haploid genome each. The diluting nucleic acid sample can have a known number of copies of the target sequence (e.g., 1, 2, 3, 4, 5, or more copies per haploid genome). In some cases, the diluting nucleic acid sample has no copies of the target sequence. In some cases, the diluting nucleic acid is isolated from cancer cells.

Any amount of diluting nucleic acid sample can be combined with any amount of query nucleic acid sample. In some embodiments, a quantity of the diluting nucleic acid sample is combined with a quantity of the query nucleic acid sample such that the ratio of the copy number of the target sequence to the copy number of the reference sequence is about 0.001, about 0.005, about 0.01, about 0.05, about 0.1, about 0.5, about 1, about 5, about 10, about 50, about 100, about 500, or about 1000. In some embodiments, a quantity of the diluting nucleic acid sample is combined with a quantity of the query nucleic acid sample such that the ratio of the copy number of the target sequence to the copy number of the reference sequence is at least about 0.001, at least about 0.005, at least about 0.01, at least about 0.05, at least about 0.1, at least about 0.5, at least about 1, at least about 5, at least about 10, at least about 50, at least about 100, at least about 500, or at least about 1000. In some cases, a quantity of the diluting nucleic acid sample is combined with a quantity of the query nucleic acid sample such that the ratio of the copy number of the target sequence to the copy number of the reference sequence is at most about 0.001, at most about 0.005, at most about 0.01, at most about 0.05, at most about 0.1, at most about 0.5, at most about 1, at most about 5, at most about 10, at most about 50, at most about 100, at most about 500, or at most about 1000. In some cases, a quantity of the diluting nucleic acid sample is combined with a quantity of the query nucleic acid sample such that the ratio of the copy number of the target sequence to the copy number of the reference sequence is between about 0.001 and about 1000, between about 0.01 and about 100, between about 0.1 and about 10, or between about 0.5 and about 5.

Methods described herein may effectively lower the copy number (“cn”) of the target locus, allowing the user to load a mixture of nucleic acid that puts the average copies/droplet closer to the statistical “sweet” spot (or target point) for both target and reference loci. In some cases, the sweet spot is about 1600 copies per microliter. A diluting nucleic acid can be added such that the copy number of the target and/or reference sequence is within about 1%, about 3%, about 5%, about 10%, about 20%, about 30%, or about 50% of 1600 copies per microliter. In some cases, the sweet spot is about 1.6 copies per droplet. A diluting nucleic acid can be added such that the copy number of the target and/or reference sequence is within about 1%, about 3%, about 5%, about 10%, about 20%, about 30%, or about 50% of 1.6 copies per droplet.

In some situations, the statistical sweet spot is a concentration for which minimal Poisson statistical uncertainty may exist. In some embodiments, a quantity of the diluting nucleic acid sample is combined with a quantity of the query nucleic acid sample such that the copy number of the target sequence and the copy number of the reference sequence are within a certain percentage of the minimal Poisson statistical uncertainty. In some cases, the copy numbers are within about 50%, about 40%, about 30%, about 20%, about 10%, about 5%, or about 1% of the minimal Poisson statistical uncertainty. In some cases, the copy numbers are within at most about 50%, at most about 40%, at most about 30%, at most about 20%, at most about 10%, at most about 5%, or at most about 1% of the minimal Poisson statistical uncertainty. In some cases, the dilution places the number of target and reference copies exactly at the statistical sweet spot for Poisson, rather than a percentage away from it.

Methods described herein may benefit from precise quantification of diluting nucleic acid to query nucleic acid. In some examples, two reference assays can be used. In another example, one of the reference assays is specific to the diluting nucleic acid sequence. In some cases, the reference assay is not specific or not found in the query nucleic acid sequence. In some cases, one of the reference assays is specific to the query nucleic acid sequence. In some instances, the reference assay is not specific or not found to the diluting nucleic acid sequence.

Methods, Devices and Systems for Sample Preparation and/or Detection

Methods for the detection of nucleic acid copy number described herein may be implemented with the aid of droplet systems. A droplet system can include a droplet generator for generating droplets, a thermal cycler for inducing nucleic acid amplification, and a droplet detector for detecting amplified nucleic acid in droplets. Following nucleic acid detection, copy number variation can be calculated using methods described above and elsewhere herein.

In an example, a system for nucleic acid analysis comprises a droplet generator, a thermal cycler and a droplet detector. The droplet generator can be used to generate droplets that may contain a sample or partition thereof. The droplets are then directed to the thermal cycler, which cycles the temperature of the droplets to induce nucleic acid amplification (e.g., PCR). Next, the droplets are directed to the droplet detector that is used to detect an amplified nucleic acid sample or partition thereof in the droplets.

Example methods and systems for generating and detecting droplets are provided herein. In some examples, a system for generating droplets comprises a first channel in fluid communication with a carrier fluid reservoir and a second channel in fluid communication with a sample reservoir. The sample reservoir can include reagents for nucleic acid amplification (e.g., PCR). The first channel and second channel meet at an intersection that is configured to generate droplets. In some cases, the droplets flow along a droplet channel to a collection reservoir. As an alternative, the droplets flow along the droplet channel to a heating zone that cycles the temperature to induce nucleic acid amplification. As another alternative, droplets from the collection reservoir are directed to a thermal cycler, which cycles the temperature of the droplets to induce nucleic acid amplification.

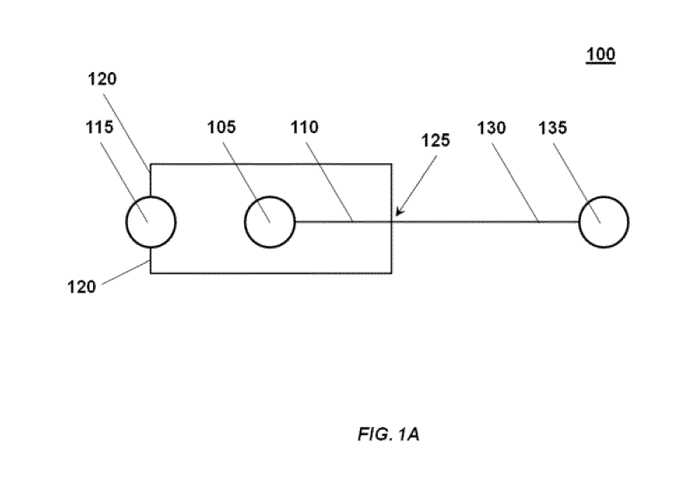

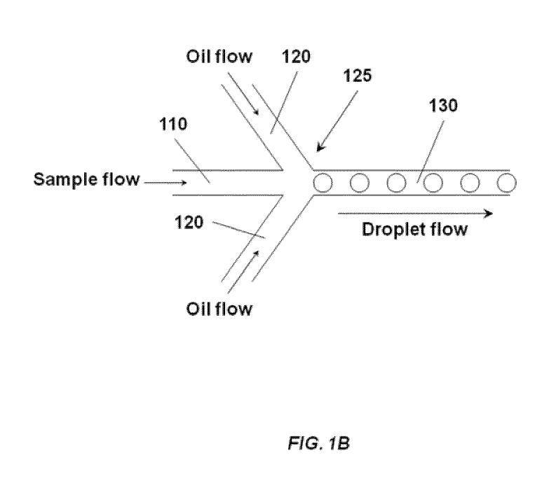

FIG. 1A shows a droplet generator 100 having a sample reservoir 105 in fluid communication with a sample channel 110, and a carrier fluid reservoir 115 in fluid communication with carrier fluid channels 120. The sample channel 110 and carrier fluid channels 120 meet at a droplet generation point (or intersection) 125. With reference to FIG. 1B, during operation, a carrier fluid (e.g., oil) from the carrier fluid reservoir 115 is directed through the carrier fluid channels 120 to the intersection 125, and a sample from the sample reservoir 105 is directed through the sample channel 110 to the intersection 125, wherein a sample partition, including any processing reagents (e.g., primers, polymerase, dyes) that may be provided from the sample reservoir 105, generate a droplet comprising an aqueous phase in an oil phase. A droplet thus formed flows in a droplet channel 130 from the intersection 125 to a droplet reservoir 135 for holding the droplets. The direction of flow of the sample, oil and droplets are indicated in FIG. 1B.

The sample channel 110 can be perpendicular or non-perpendicular to the carrier fluid channels 120. In some cases, a carrier fluid channel 120 is at an angle from about 10° to 90°, or 25° to 80°, or 40° to 70° with respect to the sample channel 110, or at least about 10°, 15°, 20°, 25°, 30°, 40°, 50°, 60°, 70°, 80°, or 85° with respect to the sample channel 110.

In some cases, a detection system can be provided along the droplet channel 130 to aid in detecting one or more samples in the droplets. In some cases, the detection system includes an excitation light source and a detector for detecting light emitted from a droplet following excitation. In still other cases, the detection of the droplets may occur at a point downstream, such as after the droplets have exited the droplet channel. For example, the detection may occur in a droplet reservoir or after the droplets exit the droplet reservoir.

The droplet generator 100 can be formed in a single-piece or multi-piece substrate. The substrate can include 1, 2, 3, 4, 5, 6, 7, 8, 9, 10, 20, 25, 30, 40, 45, 50, 100, 500, or more droplet generators. In some cases, the substrate is a consumable cassette (or cartridge) that is configured to be inserted and removed from a system for droplet generation.

Droplets with amplified nucleic acid molecules can subsequently be directed to a system for sample detection. In some examples, a system for sample detection comprises a first channel in fluid communication with a carrier fluid reservoir and a second channel in fluid communication with a sample reservoir. The first channel and second channel meet at an intersection that receives a sample from the sample reservoir and a carrier fluid (e.g., oil) from the carrier fluid reservoir and generates an emulsion that includes one or more droplets. Alternatively, an emulsion may already be formed in the sample reservoir and/or carrier fluid reservoir. In some cases, the sample in the sample reservoir may be in the form of an emulsion or a slurry. The emulsion flows along a detection channel to a collection reservoir. Flow of the emulsion is facilitated with the aid of negative pressure (or vacuum) provided at a point downstream of the intersection and/or positive pressure provided in one or more of the collection reservoir, the carrier fluid reservoir and the sample reservoir.

The system for sample detection can further comprise a detection assembly in optical communication with at least a portion of the detection channel. The detection assembly is configured to detect a signal from the droplet, such as an optical signal that may be generated upon exposure of the droplet to a source of excitation energy (e.g., excitation light).

Droplets collectively or each can include one or more dyes. In some examples, a droplet can include at least 2, 3, 4, 5, 6, 7, 8, 9, 10, 15, 20, 30, or 40 different dyes. In some embodiments, droplets can be of at least two types, such as two or more types of test droplets, test droplets and calibration droplets, or test droplets and control droplets, among others. In some embodiments, the two or more types of droplets may be distinguishable based on distinct temporal positions of the droplet types in a flow stream (or distinct times of exit from the intersection, the presence of respective distinct dyes in the droplet types, distinguishable signal intensities of the same dye (or different dyes), or a combination thereof).

In some situations, a droplet flows through a fluid flow path as an emulsion, which may be characterized by the predominant liquid compound or type of liquid compound in separate phases. For example, the phases may be an oil phase and an aqueous phase. In some cases, one or more of the phases may be a fluorous phase. In some situations, the predominant fluids in the emulsion are aqueous and oil. Oil is any liquid compound or mixture of liquid compounds that is immiscible with water that may be miscible with organic species such as alcohols and ethers. Oil may, for example, comprise a carbon and/or hydrogen content, may be non-polar, and/or may be flammable. In some examples, oil may also have a high content of fluorine, silicon, oxygen, or any combination thereof, among others. For example, any of the emulsions disclosed herein may be water-in-oil (W/O) emulsion, i.e., aqueous droplets in a continuous oil phase. Conversely, any of the emulsions disclosed herein may be oil-in-water (O/W) emulsions. This disclosure also provides multiple emulsions. For example, aqueous droplets may be enveloped by a layer of oil and flow within an aqueous continuous phase. The oil may, for example, be or include at least one of silicone oil, mineral oil, fluorocarbon oil, vegetable oil, or a combination thereof, among others. Any other suitable components may be present in any of the emulsion phases, such as at least one surfactant, reagent, sample (i.e., partitions thereof), other additive, label, particles, or any combination thereof.

Systems provided herein may be configured for use with various types of samples, such as nucleic acid samples, proteomic samples, small-molecule samples, and cellular samples. Nucleic acid samples can include deoxyribonucleic acid (DNA) and ribonucleic acid (RNA), including variants thereof (e.g., circular DNA or RNA, single-stranded DNA or RNA).

In the detection assembly, signals, such as optical (e.g., fluorescence) signals, can be detected from the droplets. The signals may include test signals, calibration signals, control signals, reference signals, or any combination thereof. In some embodiments, test signals and control signals may indicate respectively whether amplification of a test nucleic acid target and a control nucleic acid target occurred in individual droplets. In some embodiments, the detection assembly includes a detection system for collecting light and, in some cases, providing excitation energy, such as excitation light. The wavelength of excitation light can be selected to induce excitation within a droplet.

Detection in the detection assembly may include (a) exciting a dye with the aid of excitation light and (b) detecting emitted light from the dye. In some embodiments, detection in the detection assembly includes (a) exciting multiple dyes with of the aid of excitation light and (b) detecting emitted light from the dyes at least substantially independently from one another in one or more detector channels.

The system for sample detection may further include a third channel in fluid communication with the carrier fluid reservoir. The third channel may meet with the first and second channels at the intersection.

In some examples, one or more samples from the sample reservoir are directed to the intersection in droplets and brought in contact with a carrier fluid from the carrier fluid reservoir to form an emulsion having the droplets. Alternatively, the sample reservoir may supply a pre-formed emulsion of sample droplets. An individual droplet may include a sample or sample partition. The droplets flow along the detection channel as an emulsion that may be made up of a plurality of phases, such as a first phase and a second phase. The first and second phases may be separated by an outer boundary of the droplet, such as a skin.

The droplets, skin, or both may be formed prior to forming the emulsion. In some examples, a droplet is formed in a separate or integrated droplet generator (see above). The droplet may include a skin, which may also be formed in the droplet generator.

In some embodiments, the droplet detector is included in a housing having one or more droplet detectors. The housing can include a plurality of droplet detectors for parallel detection, which can aid in maximizing detection efficiency—e.g., a plurality of samples can be detected in parallel, thereby reducing droplet detection time.

In some embodiments, the system includes a pressure source for facilitating the flow of droplets from the intersection to the collection reservoir. The pressure source can be a source of positive pressure operatively coupled to the carrier fluid and/or sample reservoir, or a source of negative pressure (i.e., vacuum) operatively coupled to the fluid flow path, such as by way of the collection reservoir. The source of positive pressure can be a compressor or a pressurized fluid, such as a pressurized gas (e.g., pressurized air). The source of negative pressure can be a pumping system comprising one or more pumps, such as mechanical pumps.

The system may be configured for nucleic acid amplification, such as polymerase chain reaction (PCR). In some embodiments, an energy providing device is used to raise the temperature of droplets provided at the intersection to initiate amplification. The system can thermally cycle the temperature of the droplets, from a low temperature to a high temperature, and in some cases to a low temperature with the aid of cooling. Cooling can be implemented with the aid of heat fins, for instance, or a cooling system, such as a thermoelectric cooling system.

In some situations, the system includes a detection assembly in fluid communication with the fluid flow path. The detection assembly may be situated along at least a portion of the detection channel between the intersection and the collection reservoir. The detection assembly can be configured to detect signals from droplets in the fluid flow path, such as upon flowing through the detection channel. The detection assembly can include an optical sensor or other electronic detector that is sensitive to a select frequency of light. The sensor can be adapted to detect fluorescent emission, for example. In some cases, the detection assembly can include an excitation source, such as a light source that is adapted to induce fluorescence in the fluid. One or more optical elements (e.g., mirrors, lenses) can be provided to direct light emitted from the fluid to the detection assembly, and/or to direct light from a light source to the fluid.

In some examples, a device for sample detection comprises a first channel in fluid communication with a carrier fluid reservoir and a second channel in fluid communication with a sample reservoir. The first channel and second channel can meet at an intersection to form an emulsion upon the carrier fluid coming in contact with the sample. Alternatively, an emulsion comprising droplets may already be formed in the sample reservoir and/or carrier fluid reservoir. The emulsion can include droplets containing a sample from the sample reservoir. In some cases, the emulsion may be formed separately and/or off-line and be provided to the device (e.g., by pipetting). The sample reservoir may comprise the droplet(s).

The device may further include a detection channel leading from the intersection to a collection reservoir. The emulsion may flow along the detection channel to the collection reservoir. A detection assembly may be in optical communication with at least a portion of the detection channel. The detection assembly may be adapted to detect an electromagnetic (i.e., optical) signal from the droplet. In some instances, at least a portion of the detection channel includes sample amplification (e.g., PCR amplification of a nucleic acid).

In some examples, a detection assembly may be arranged in a stop-flow configuration. In such a configuration, droplets are captured in non-flow conditions and detected. For example, droplets may be individually arrayed on a slide or each entered into an individual well of a multi-well plate. The slide or multi-well plate comprising the droplets may then be brought into communication with a detection assembly for detection of the droplets.

Emulsions and/or droplets may be maintained at a constant temperature in the devices, systems, and methods described herein. Constant temperature can be provided by heating the droplets and/or emulsions and may be advantageous for obtaining an accurate signal in an assay. A constant temperature may vary by less than about 10° C., less than about 1° C., less than about 0.5° C., or less than about 0.1° C. Systems of the disclosure can have a coefficient of variation (CV) less than about 20%, 15%, 10%, 5%, 4%, 3%, 2%, 1%, 0.1%, or less.

The detection assembly may be disposed along the detection channel. The detection assembly may detect any suitable signal from the droplets. In some cases, the detection assembly includes confocal optics. The detection assembly may be in optical communication with a source of energy (e.g., visible light).

The device may further comprise a pressure source for facilitating the flow of the droplet to the collection reservoir. The pressure source may include a source of positive pressure or negative pressure.

The device may further comprise a controller in communication with the detection assembly. The controller can include a computer processor programmed to estimate the presence or absence of a nucleic acid target in the sample.

In some examples, the intersection is a singulator that receives a carrier fluid (e.g., focusing oil) and droplets from a sample reservoir comprising the droplets. The singulator can separate droplets prior to detection by a detection assembly.

The sample reservoir may comprise one or more droplets. An individual droplet may comprise a sample or sample partition, such as a nucleic acid sample.

Methods of the present disclosure may further comprise flowing the emulsion along the detection channel. The emulsion may flow to a collection reservoir or to a system or sub-system downstream of the detection channel, such as a detection assembly. As an alternative, the detection assembly can be coupled to at least a portion of the detection channel. The detection assembly may include a source of excitation energy and a detector for detecting a signal emitted from a droplet upon excitation with the excitation energy.

In some examples, a carrier fluid from a carrier fluid reservoir and droplet (or plurality of droplets) in a sample reservoir are induced to flow to the intersection with the aid of positive pressure supplied to the sample reservoir and/or the carrier fluid reservoir, or negative pressure (vacuum) supplied to the collection reservoir upstream of the intersection. An emulsion comprising the droplets may be formed at the intersection or may already be formed upstream from the intersection. In some cases, both positive and negative pressure are used to facilitate the flow of fluid to the intersection and subsequent flow of an emulsion comprising the droplet(s) to the collection reservoir. The emulsion is directed along a detection channel through a detection zone coupled to, or part of, a detection assembly. The droplet(s) and/or sample (or sample partition) are then detected with the aid of the detection assembly. The droplet(s) is then directed to the collection reservoir.

In some situations, upon flow of the emulsion along the detection channel, a signal from a droplet in the emulsion may be detected. The signal may be an electromagnetic (or optical), electrostatic, electrochemical, or magnetic signal. In some examples, an optical signal is detected. The optical signal may be from the droplet in a detection assembly in optical communication with at least a portion of the detection channel. The optical signal may be generated by directing excitation energy (e.g., excitation light) into the emulsion and detecting a signal emitted from the emulsion upon excitation. The signal may be a fluorescence signal from a dye associated with a sample or sample partition in a droplet, such as, for example, an intercalated dye.

The detection system may include a droplet generator upstream of the detection assembly. The droplet generator may be separately situated in relation to the detection assembly, such as in different systems. As an alternative, the droplet generator and the detection assembly (or droplet detector) are part of the same system and may be in fluid communication with one another.

In some cases, as a droplet flows from the intersection to the collection reservoir or a detection region along the detection channel, the temperature of the detection channel may be cycled to induce nucleic acid amplification. This can advantageously enable in-line nucleic acid amplification prior to sample detection. In some cases, prior to temperature cycling, the droplet is heated to induce skin formation around the droplet.

In some cases, droplets are exposed to a sequence of temperatures to enable a PCR reaction (e.g., a denaturation temperature, an annealing temperature, and an extension temperature) prior to detection. The temperatures may be optimized for a particular assay. Examples of temperatures may be from about 94° C. to 96° C. Examples of annealing temperatures may be from about 37° C. to 75° C. Examples of extension temperatures may be from about 60° C. to 72° C. In some cases, the droplets are exposed to temperature to enable hot-start of an enzyme, such as a polymerase. An example temperature for enabling hot-start is about 95° C.

In some situations, a droplet is stabilized by heating (e.g., incubating) the droplet at a temperature between about 4° C. and 99° C., or 30° C. and 80° C., or 50° C. and 65° C. for at least 0.1, 1, 2, 3, 4, 5, 6, 7, 8, 9, 10, 20, 30, 40, 50, 60, 70, 80, 90, 100, 200, 300, 400, 500, or 1000 seconds. In some examples, the droplet may be stabilized by heating the droplet at a temperature between about 50° C. and 65° C. for 5 or more seconds. In other examples, the droplet is stabilized by heating the droplet at a temperature between about 30° C. and 80° C. for a time period between about 5 seconds and 2 hours. In other examples, the droplet is stabilized by heating the droplet at a temperature between about 80° C. and 95° C. for a time period between about 5 seconds and 30 minutes.

During or prior to detection, a droplet may be heated along a temperature gradient. The temperature gradient may have a first temperature at a first portion of the detection channel and a second temperature at a second portion of the detection channel downstream of the first portion. The temperature gradient can have temperatures from about 55° C. and 98° C. In an example, the temperature at the first portion is 55° C. and the temperature at the second portion is 75° C., and the temperature from the first portion to the second portion is increased (e.g., gradually increased) from 55° C. to 75° C. Alternatively, the droplet can be heated at a constant temperature for a time sufficient to minimize or eliminate the signal generated from a non-specific target. In an example, the droplet is heated at a temperature from about 55° C. and 98° C. for a time period from about 1 second to 15 minutes.

In some cases, methods may include the detection of a nucleic acid in a sample. Such methods may comprise (a) providing a sample comprising a plurality of partitions, wherein at least one of the partitions comprises an amplified nucleic acid, and (b) detecting an optical signal from at least one of the partitions, wherein the temperature of the partition is at least 50° C. The optical signal can be correlated with an amount of the nucleic acid.

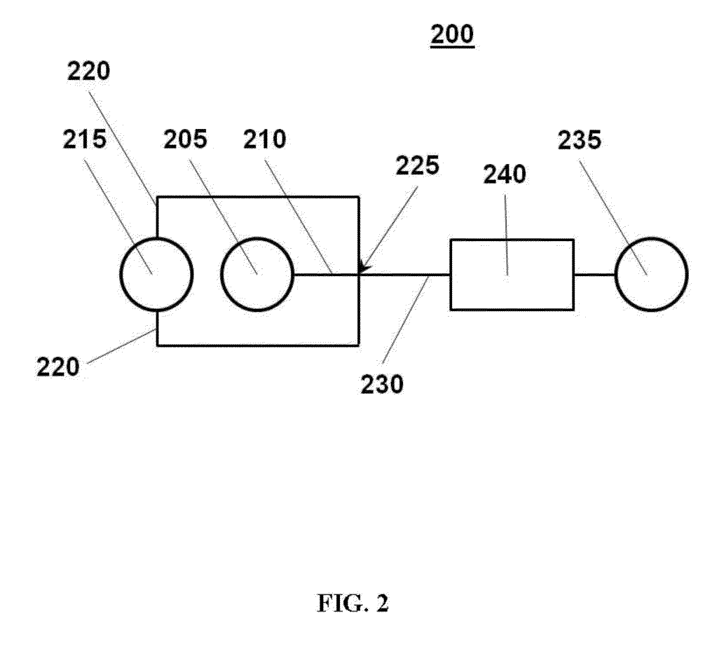

FIG. 2 shows an example droplet detection system 200. The detection system 200 includes a sample reservoir 205 in fluid communication with a sample channel 210 and a carrier fluid reservoir 215 in fluid communication with carrier fluid channels 220. The sample channel 210 and carrier fluid channels 220 meet at an intersection 225. During operation, a carrier fluid (e.g., oil) from the carrier fluid reservoir 215 is directed through the carrier fluid channels 220 to the intersection 225, and a sample (e.g., a sample in a droplet) from the sample reservoir 205 is directed through the sample channel 210 to the intersection 225. The carrier fluid and the sample may be directed with the aid of positive and/or negative pressure. At the intersection 225, an emulsion may be generated comprising the carrier fluid and a sample or sample partition, such as one or more droplets each comprising a sample or sample partition. Alternatively, an emulsion may already be formed in sample reservoir 205 prior to sample arrival at intersection 225. In some examples, the emulsion comprises a droplet in the carrier fluid. An emulsion then flows in a detection channel 230 from the intersection 225 to a collection reservoir 235. A detection assembly 240 along the detection channel 230 detects droplets in the emulsion as the emulsion flows from the intersection 225 to the collection reservoir 235. The detection assembly 240 may be a droplet detector, as described elsewhere herein.

The sample channel 210 may be perpendicular or non-perpendicular to the carrier fluid channels 220. In some cases, a carrier fluid channel 220 is at an angle from about 10° to 90°, or 25° to 80°, or 40° to 70° with respect to the sample channel 210, or at least about 10°, 15°, 20°, 25°, 30°, 40°, 50°, 60°, 70°, 80°, or 85° with respect to the sample channel 210.

The detection assembly 240 may include one or more components (e.g., optics, sensors) for detecting a signal emanating from a droplet. In some cases, the detection system includes an excitation light source and a detector for detecting light emitted from a droplet following excitation. The detection assembly 240 may be coupled to one or more detection regions of the detection channel 230 (e.g., through a channel or capillary). In some examples, the one or more detection regions of the detection channel 230 include windows for permitting an electromagnetic (or optical) signal to reach a fluid (e.g., emulsion), that may include one or more droplets, flowing through the detection channel. The detection assembly 240 may be a droplet detector, as described in, for example, U.S. Patent Publication No. 2010/0173394 to Colston et al. (“Droplet-based assay system”), which is entirely incorporated herein by reference for all purposes.

The sample reservoir 205 can include droplets having samples or sample partition therein. Each droplet can include a nucleic acid sample or portion thereof, and a species that is configured to be excited by a source of excitation energy or stimulus. Some examples of species that are configured to be excited include dyes, such as intercalating dyes or labeled probes, such as labeled oligonucleotide probes. Examples of intercalating dyes are ethidium bromide, SYBR Green™, SYBR Gold™, 4′,6-diamidino-2-phenylindole (DAPI), or combinations thereof. A labeled oligonucleotide probe may be, for example, a TaqMan probe, wherein quenched flurophores labels bound to the oligonucleotide probe are released by the exonuclease activity of a DNA polymerase (e.g., Taq polymerase) after probe binding to its target. Release of the fluorophore from quenching may result in its detection. Moreover, a droplet may have a skin on an outer portion of the droplet. The skin may aid in providing droplet stability during detection.

An individual droplet can include a sample or sample partition. The sample or sample partition may be a nucleic acid sample (e.g., DNA or RNA sample), which may have been amplified, such as with the aid of polymerase chain reaction (PCR). As an alternative, the individual droplet may include reagents (e.g., primers, polymerase(s), nucleic acids) for nucleic acid amplification.

The system of FIG. 2 may be integrated with a droplet generator, such as droplet generators described in U.S. Patent Publication No. 2010/0173394 to Colston et al. (“Droplet-based assay system”), which is entirely incorporated herein by reference for all purposes.

In some cases, an associated droplet generator may be separately situated in relation to a droplet detector. In other cases, the droplet generator and the droplet detector are part of the same system and may be in fluid communication with one another. For example, one or both of the sample reservoir 205 and the carrier fluid reservoir 215 of the system 200 of FIG. 2 may be precluded or modified to account for a device having a droplet generator upstream of the system of FIG. 2.

The systems of FIG. 2 can be formed in a single-piece or multi-piece substrate. The substrate can include at least 1, 2, 3, 4, 5, 6, 7, 8, 9, or 10 droplet detectors, each of which may be as described in FIG. 2.

Droplets may have skins, which may be formed in a droplet generator before the droplet, including a sample in the droplet, is detected with the aid of the detection assembly. In some cases, a droplet with a skin is capable of withstanding shear forces or other mechanical perturbations for a time period of at least about 1 second, 10 seconds, 30 seconds, 1 minute, 10 minutes, 30 minutes, 1 hour, 2 hours, 3 hours, 4 hours, 5 hours, 6 hours, or more. Various approaches for droplet stabilization, including the use of droplets with skins, are described in U.S. patent application Ser. No. 14/018,205, filed Sep. 4, 2013, which is entirely incorporated herein by reference for all purposes.

The path of a detection channel can be substantially linear. In some embodiments, the path of the detection channel can comprise one or more meanders. A meander can be a section of fluid flow path that does not take the shortest path between two points. The one or more meanders can be in series, in parallel, or a combination of series and parallel. The meanders can be configured to provide a desired or otherwise predetermined flow resistance, a desired residence time, a desired mixing, or the like. In some examples, a meander can be configured to provide a residence time for an emulsion to equilibrate to a desired or otherwise given temperature. In some examples, a meander can be configured to provide a residence time for an emulsion to incubate for a given time at a given temperature.

A droplet may flow through a detection channel at any suitable rate. The flow rate can be equal to droplets per time multiplied by the average droplet volume. In some examples, one or more droplets flow at a rate of about 0.1, about 0.5, about 1, about 5, about 10, about 50, about 100, about 500, about 1000, about 5000, about 10000, or about 50000 μl/minute. In some cases, one or more droplets flow at a rate of at least about 0.1, at least about 0.5, at least about 1, at least about 5, at least about 10, at least about 50, at least about 100, at least about 500, at least about 1000, at least about 5000, at least about 10000, or at least about 50000 μl/minute. One or more droplets may flow at a rate of between 0.5 μl/minute and 10000 μl/minute, or between 1 μl/minute and 5000 μl/minute. Energy may be provided to one or more droplets under flow.

In some examples, one or more droplets flow along a detection channel at a flow rate between about 0.5 microliter/minute and 10,000 microliters/minute, or 1 microliter/minute and 5,000 microliters/minute. The flow rate can be computed by the relationship: number of droplets/time*average volume (μl)/droplet. Energy may be provided to the one or more droplets under flow.

The Weber number is a dimensionless number that is often useful in analyzing fluid flows where there is an interface between two different fluids, such as multiphase flows with strongly curved surfaces (e.g., emulsions). The Weber number can be thought of as the relative importance of the fluid's inertia compared to its surface tension. The Weber number is the density of the fluid multiplied by the square of its velocity multiplied by the droplet diameter divided by the surface tension.

The Weber number associated with the emulsion directed through the detection channel may be about 0.01, about 0.05, about 0.1, about 0.5, about 1, or about 5. In some cases, the Weber number is at least about 0.01, at least about 0.05, at least about 0.1, at least about 0.5, at least about 1, or at least about 5. In some situations, the Weber number may be at most about 0.01, at most about 0.05, at most about 0.1, at most about 0.5, at most about 1, or at most about 5.

The Reynolds number is a dimensionless number that is a measure of the ratio of inertial forces to viscous forces. In some cases, the Reynolds number is calculated by multiplying the density of the fluid with the mean velocity of the object relative to the fluid times a characteristic linear dimension and dividing by the dynamic viscosity of the fluid.

In some instances, the flow through the detection channel is laminar (e.g., has a Reynolds number that is less than about 2100). In some instances, the flow is turbulent (e.g., has a Reynolds number that is greater than about 2100). In some embodiments, the Reynolds number is about 0.05, about 0.1, about 0.5, about 1, about 5, about 10, about 50, about 100, about 500, about 1000, about 5000 or about 10000. In some embodiments, the Reynolds number is at least about 0.05, at least about 0.1, at least about 0.5, at least about 1, at least about 5, at least about 10, at least about 50, at least about 100, at least about 500, at least about 1000, at least about 5000 or at least about 10000. In some embodiments, the Reynolds number is at most about 0.05, at most about 0.1, at most about 0.5, at most about 1, at most about 5, at most about 10, at most about 50, at most about 100, at most about 500, at most about 1000, at most about 5000 or at most about 10000. In some embodiments, the Reynolds number is between 0.1 and 1000.

In some examples, an individual droplet flows at Weber number of 1 or less. The Reynolds number of an individual droplet in an emulsion, or a plurality of droplets in the emulsion, may be less than about 2100, or in some cases greater than 2100. In some cases, the Reynolds number is between 0.1 and 1000.

In some examples, a droplet or emulsion comprising the droplet is heated by heating an oil in the carrier fluid reservoir such that the oil, upon flowing from the carrier fluid reservoir to the intersection, has a Reynolds number of at least about 1, 10, 1000, 2000, 3000, 4000, 5000 or higher.

This disclosure provides detection assemblies adapted to detect samples in droplets. A detection assembly may be adapted to detect an electromagnetic signal from the droplet. A detection assembly may be coupled to the detection channel. The detection channel may include a capillary for directing droplets to a detection region in communication with the detection assembly.

In some examples, the detection assembly includes an electromagnetic energy source and an electromagnetic energy detector, such as, for example, a fluorescence detector, which may be suited to detect fluorescence emissions from a droplet. The electromagnetic energy source and the electromagnetic energy detector may be used to irradiate, track, and analyze droplets. An electromagnetic energy detector may include a forward scatter detector. The electromagnetic energy source may provide excitation electromagnetic energy that has a frequency or range of frequencies for exciting an excitable species coupled to a sample in a droplet, such as a dye (e.g., fluorescence dye). The detection assembly can include optics (e.g., lenses, mirrors), which may direct the excitation electromagnetic energy to a droplet comprising a sample. Following excitation, the excitable species may emit an electromagnetic signal that may be detected by the electromagnetic energy detector. Optics may be provided for directing emitted electromagnetic energy to the detector.

Methods described herein can be used separately or in combination. In some cases, diluting nucleic acid is added to a sample. In some embodiments, diluting nucleic acid is not added to the sample and the copy number is determined using any one of the methods described herein.

In an aspect, the present disclosure provides a method for determining copy number in a digital PCR assay that includes serial dilutions and multiple wells. A sample can be split into a target portion and a reference portion. The reference portion can be tested to determine the amount of a reference gene in the sample. The target portion can be first diluted, in some instances to reduce its concentration to a level similar to the reference, then tested to determine the amount of target gene in the sample.

In another aspect, the present disclosure provides a method for determining copy number in a digital PCR assay that includes measuring the reference gene at multiple locations and/or measuring multiple reference genes. For instance, if the reference gene is measured at 10 locations and a target has a copy number of 50, then the dynamic range of the digital analysis system would be about 5 instead of 50. In some cases, the sample is separated into multiple locations isolated from one another (e.g., by a restriction digest) to get a readout for each reference location.

In another aspect, the present disclosure provides a method for determining copy number in a digital PCR assay that includes measuring one or more moderately elevated copy number genes (having a copy number between the target and reference) along with the target and reference. In some cases, two or more genes are selected that have a range of copy numbers distributed between the target and the reference gene. In the present example, one gene of intermediate copy number is used. The target is referred to as T, the moderately elevated copy number gene is referred to as G and the reference is referred to as R. In a first measurement, the amount of G is measured relative to the reference R. The ratio of G/R is preferably within the dynamic range of the digital analysis system. In a second measurement, the amount of T relative to the amount of G is measured in a diluted sample. The ratio of T/G is preferably within the dynamic range of the digital analysis system. In this case, the copy number of the target (CN) can be estimated as CN=(G/R)*(T/G).

In another aspect, the present disclosure provides a single-well method for determining copy number in a digital PCR assay. When a sample has a high target copy number (e.g., 100 copies of the target for every one copy of the reference), the sample can be screened at the DNA load where the reference is at 1.6 copies per droplet, then diluted 100-fold so the target can be loaded at 1.6 copies per droplet. In some case, software can provide the dilution number, can compare the reactions in each well, can factor in the dilution, and can report back an accurate copy number for the target. This method relies on two reactions requiring a single detection channel.

Computer Systems for Calculating Copy Number

The present disclosure provides computer systems for calculating copy number and determining copy number variation. The computer system can be programmed or otherwise configured to operate with droplet-based nucleic acid sample detection methods described herein.

The number of copies of a given nucleic acid sequence in the nucleic acid sample is retained in computer memory of a computer system used to calculate copy number of the given nucleic acid sequence in the nucleic acid sample.

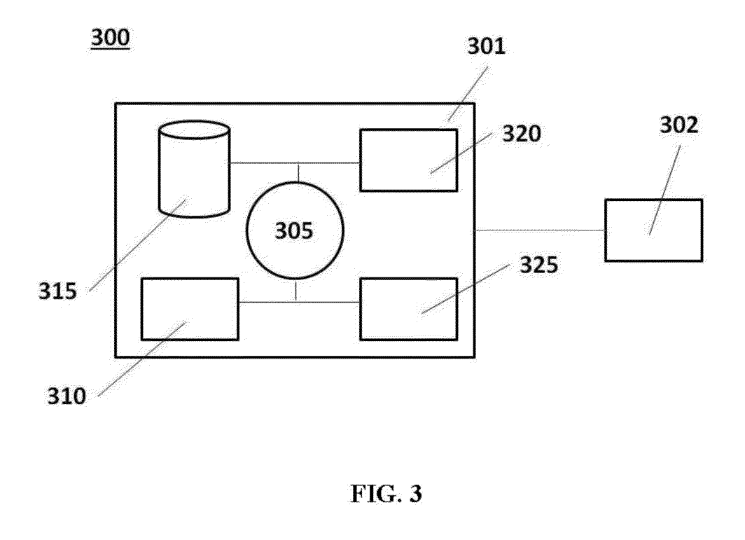

Copy number variation systems and methods of the disclosure may be operated or regulated with the aid of computer systems. Calculations may also be performed using software running on computer systems. FIG. 3 shows a system 300 comprising a computer system 301 coupled to a nucleic acid sequencing system 302. The computer system 301 may be a server or a plurality of servers. The computer system 301 may be programmed to regulate sample preparation and processing, and nucleic acid sequencing by the sequencing system 302. The sequencing system 302 may be a nanopore-based sequencer (or detector), as described elsewhere herein.

The computer system may be programmed to implement the methods of the present disclosure. The computer system 301 includes a central processing unit (CPU, also “processor” herein) 305, which can be a single core or multi core processor, or a plurality of processors for parallel processing. The computer system 301 also includes memory 310 (e.g., random-access memory, read-only memory, flash memory), electronic storage unit 315 (e.g., hard disk), communications interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communications bus (solid lines), such as a motherboard. The storage unit 315 can be a data storage unit (or data repository) for storing data. The computer system 301 may be operatively coupled to a computer network (“network”) with the aid of the communications interface 320. The network can be the Internet, an internet and/or extranet, or an intranet and/or extranet that is in communication with the Internet. The network can include one or more computer servers, which can enable distributed computing.

Methods of the present disclosure can be implemented by way of machine (or computer processor) executable code (or software) stored on an electronic storage location of the computer system 301, such as, for example, on the memory 310 or electronic storage unit 315. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine have a processer adapted to execute the code, or can be compiled during runtime. The code can be supplied in a programming language that can be selected to enable the code to execute in a pre-compiled or as-compiled fashion.

The computer system 301 can be adapted to store user profile information, such as, for example, a name, physical address, email address, telephone number, instant messaging (IM) handle, educational information, work information, social likes and/or dislikes, and other information of potential relevance to the user or other users. Such profile information can be stored on the storage unit 315 of the computer system 301.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., ROM, RAM) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Multi-channel Detection

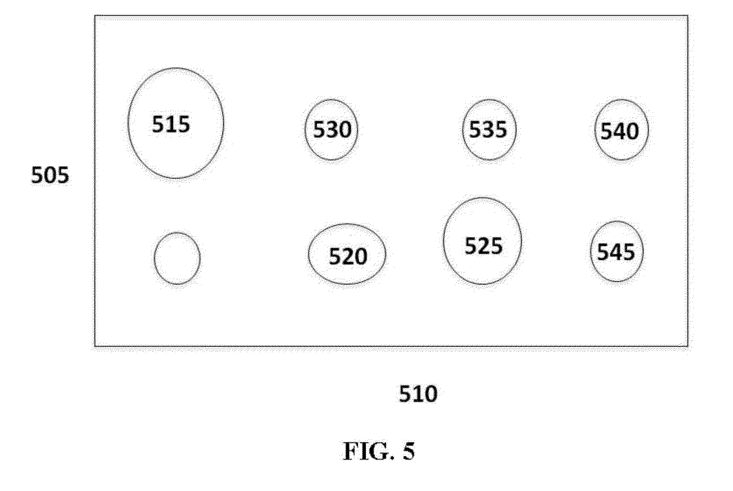

Methods of the present disclosure can be performed with any number of detectors and/or a detector having any number of channels (e.g., 1, 2, 3, 4, 5, or more). FIG. 5 shows an example of a 2-channel detector having a first channel 505 and a second channel 510. There are two separate references being detected on the second channel (e.g., R1 and R2). The assay can be performed in a manner such that the single positive droplets containing R1 are not as bright as the droplets containing R2. The method can involve clustering to quantify the amount of query versus diluting nucleic acid.

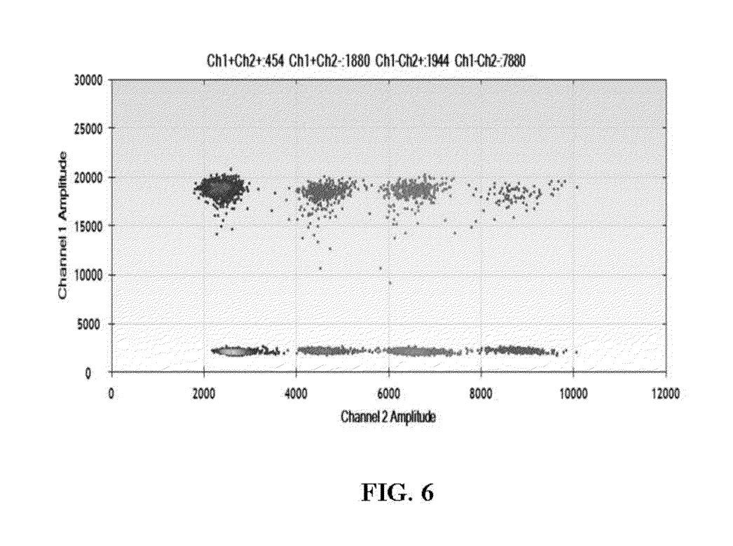

In this example, R1 is unique to the diluting nucleic acid (e.g., the cluster is smaller than for R2). In this case, the reference R2 is a sequence that is found in both the query and diluting nucleic acid, so quantifying R2 can determine how many genome equivalents are found in the sample. Double and triple positive droplets are shown. In particular, cluster 515 is the target, cluster 520 is R1, cluster 525 is R2, cluster 530 is T/R1, cluster 535 is T/R2, cluster 540 is T/R1 and R2, and cluster 545 is R1 and R2 in this example. FIG. 6 provides an example of 3-channel detection (“triplex assay”).

Systems and methods of the present disclosure may be used to sequence various types of biological samples, such as nucleic acids (e.g., DNA, RNA) and proteins. Methods, devices and systems described herein can be used to sort biological samples (e.g., cells, proteins or nucleic acids). The sorted samples and/or molecules can be directed to various bins for further analysis.

Methods of the present disclosure can enable the determination of copy number variation within a given degree of accuracy. In some cases, the measured copy number is within an accuracy (i.e., in either the positive or negative direction) of about 1%, about 3%, about 5%, about 10%, about 15%, about 20%, about 30%, about 40%, or about 50% from the true copy number.

Methods of the present disclosure can be suitable for digital analysis systems with limited dynamic range. One factor that can affect the dynamic range of a digital analysis system is the number of partitions and/or droplets where the lower the number of partitions, the lower the dynamic range. Methods of the present disclosure can be performed when the number of partitions is less than about 5000000, less than about 3000000, less than about 1000000, less than about 100000, less than about 50000, less than about 20000, less than about 10000, less than about 5000, or less than about 1000.

### EXAMPLES

### Example 1

### Determination of Copy Number

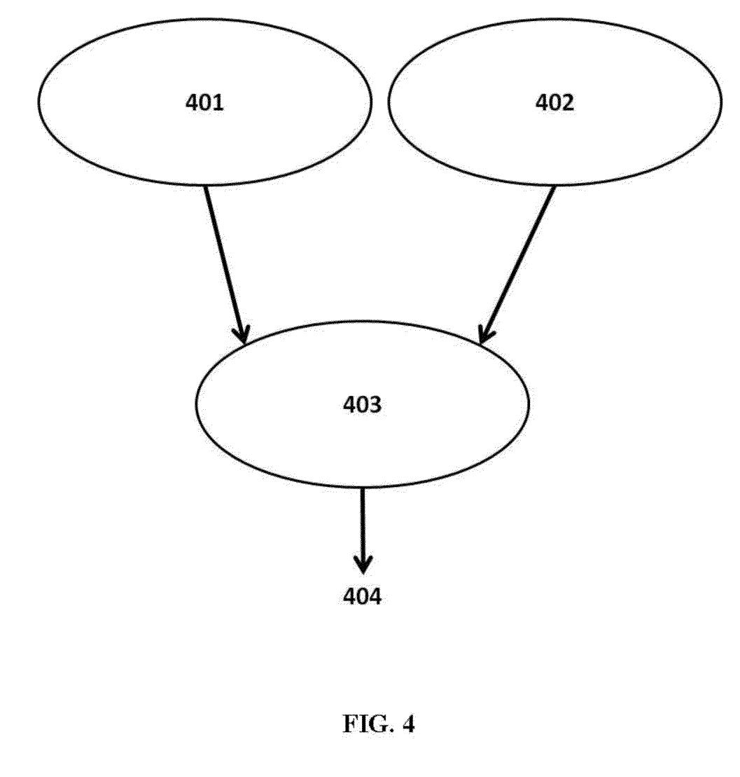

By way of illustration and without limitation, equal amounts of query nucleic acid sample and diluting nucleic acid sample are combined as shown in FIG. 4. The query nucleic acid sample 401 has 100 copies of the target sequence per one copy of the reference sequence (target:reference=100:1). The diluting nucleic acid sample 402 has one copy of the target sequence per copy of the reference sequence (target:reference=1:1). The combined nucleic acid sample 403 has approximately 50.5 copies of the target sequence per one copy of the reference sequence (target:reference=50.5:1).

Digital PCR is used to measure the relative copy number of the target sequence compared to the reference sequence in the combined sample 404. A separate assay is used to quantify how much of the diluting nucleic acid is added. Absorbance at 260 nm wavelength can be used to know how much dilution has occurred when the query nucleic acid does not have a unique sequence that can be quantified.

### Example 2

### Determination of Copy Number

By way of illustration and without limitation, query nucleic acid sample and a 10-fold excess of diluting nucleic acid sample are combined as shown in FIG. 4. The query nucleic acid sample 401 has 100 copies of the target sequence per one copy of the reference sequence (target:reference=100:1). The diluting nucleic acid sample 402 has one copy of the target sequence per copy of the reference sequence (target:reference=1:1). The combined nucleic acid sample 403 has approximately 10 copies of the target sequence per one copy of the reference sequence (target:reference=10:1).

Digital PCR is used to measure the relative copy number of the target sequence compared to the reference sequence in the combined sample 404. A separate assay is used to quantify how much of the diluting nucleic acid is added. Absorbance at 260 nm wavelength can be used to know how much dilution has occurred when the query nucleic acid does not have a unique sequence that can be quantified.

### Example 3

### Determination of Copy Number

By way of illustration and without limitation, query nucleic acid sample and a 100-fold excess of diluting nucleic acid sample are combined as shown in FIG. 4. The query nucleic acid sample 401 has 100 copies of the target sequence per one copy of the reference sequence (target:reference=100:1). The diluting nucleic acid sample 402 has one copy of the target sequence per copy of the reference sequence (target:reference=1:1). The combined nucleic acid sample 403 has approximately 1.98 copies of the target sequence per one copy of the reference sequence (target:reference=1.98:1).

Digital PCR is used to measure the relative copy number of the target sequence compared to the reference sequence in the combined sample 404. A separate assay is used to quantify how much of the diluting nucleic acid is added. Absorbance at 260 nm wavelength can be used to know how much dilution has occurred when the query nucleic acid does not have a unique sequence that can be quantified.

In some cases, the higher the amount of reference nucleic acid added, the closer to a 1:1 ratio is achieved. In some instances, the highest level of precision can be achieved if the target and query nucleic acid is loaded at the Poisson sweet spot (e.g., both at a 1:1 ratio).

### Example 4

### Determination of Copy Number

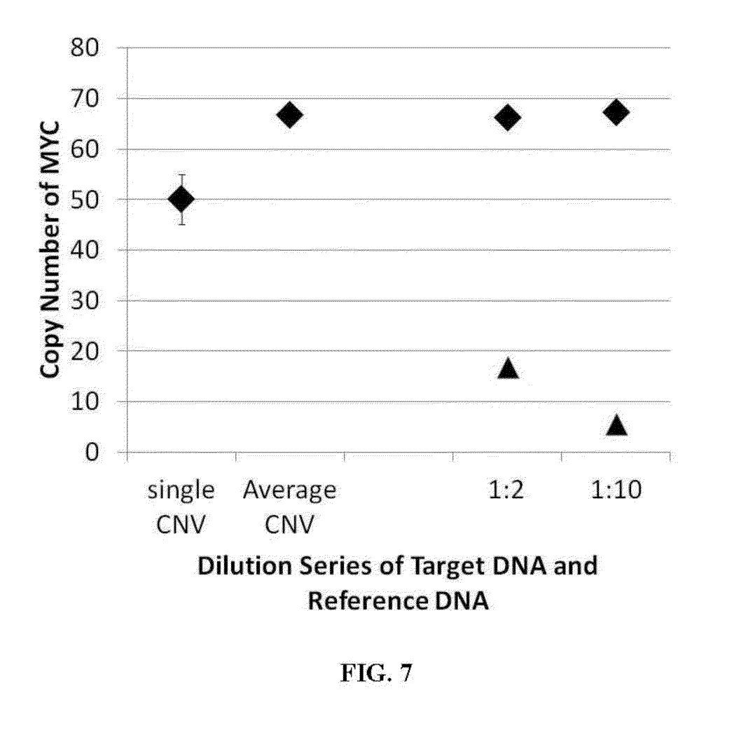

A query cell line over expressing the Myc gene is provided. A diluting cell line is added (at 1:2 and 1:10 dilution) to this query cell line. A triplex reaction is performed. The triplex reaction has an assay targeting the Myc gene, an assay targeting a conserved reference gene common to both the query cell line and the diluting cell line (termed “Fan assay”), and an assay that targets a variant that is unique to the diluting cell line (termed “1521 assay”). The degree of dilution is determined by looking at the fan and 1521 concentration measurements (as shown in FIG. 7). In this case, the Fan assay is targeting a region present at 2 copies per diploid genome, and the 1521 assay is targeting a region present at 1 copy/diploid genome, so the 1521 numbers are multiplied by 2.

The copy number of the target (CN) can be determined by the equation CN=(2*[Myc]/[Fan]−ratioRef*CNrefDNA)/ratioTar, where [MYC]=concentration of MYC; [Fan]=concentration of Fan_ch1; ratioTar=dilution factor of Target DNA in reference DNA=1−ratioRef; ratioRef=[RefS]/[RefT]; and CNrefDNA=copy number of reference assays in reference DNA.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

What is claimed is:

1. A method for determining nucleic acid copy number, the method comprising:
(a) providing a query nucleic acid sample comprising a target nucleic acid sequence and a reference nucleic acid sequence;
(b) combining, with the query nucleic acid sample, a diluting nucleic acid sample having a known copy number of the target nucleic acid sequence and the reference nucleic acid sequence; and
(c) measuring the relative copy number of the target nucleic acid sequence compared to the reference nucleic acid sequence in the combined sample using digital polymerase chain reaction (PCR).

2. The method of claim 1, wherein nucleic acid molecules in the query nucleic acid sample comprise at least one copy of the reference nucleic acid sequence and at least one copy of the target sequence.

3. The method of claim 1, wherein the digital PCR is performed in an emulsion having one or more droplets.

4. The method of claim 3, wherein the droplets comprise one copy of the target nucleic acid sequence and/or reference nucleic acid sequence.

5. The method of claim 1, wherein the quantity of query nucleic acid sample is measured or known to within an accuracy of about 5%.

6. The method of claim 1, wherein the query nucleic acid sample has at least 10-fold more copies of the target nucleic acid sequence than the reference nucleic acid sequence.

7. The method of claim 1, wherein the query nucleic acid sample has at least 10,000-fold more copies of the target nucleic acid sequence than the reference nucleic acid sequence.

8. The method of claim 1, wherein a quantity of the diluting nucleic acid sample is combined with a quantity of the query nucleic acid sample such that the ratio of the copy number of the target nucleic acid sequence to the copy number of the reference nucleic acid sequence is between about 1 and 10.

9. The method of claim 1, wherein a quantity of the diluting nucleic acid sample is combined with a quantity of the query nucleic acid sample such that the copy number of the target nucleic acid sequence and the copy number of the reference nucleic acid sequence are within about 20% of a minimal Poisson statistical uncertainty.

10. The method of claim 1, wherein the target nucleic acid sequence is a deoxyribonucleic acid sequence.

11. The method of claim 1, wherein the reference nucleic acid sequence is a deoxyribonucleic acid sequence.

12. The method of claim 1, wherein (c) is performed on three measurement channels, wherein a first channel of the three measurement channels quantifies the target nucleic acid, a second channel of the three measurement channels quantifies a total number of genome equivalents and a third channel of the three measurement channels quantifies an amount of diluting nucleic acid.

13. The method of claim 12, wherein a computer calculates the nucleic acid copy number using the three measurement channels.

14. A method for performing digital polymerase chain reaction (PCR), the method comprising:
(a) providing a query nucleic acid sample comprising a target nucleic acid sequence and a first reference nucleic acid sequence;
(b) combining with the query nucleic acid sample, a diluting nucleic acid sample having the first reference nucleic acid sequence and a second reference nucleic acid sequence;
(c) dividing the combined sample into a plurality of reaction volumes;
(d) performing a PCR reaction on the reaction volumes to determine the concentration of the target nucleic acid sequence, the first reference nucleic acid sequence and the second reference nucleic acid sequence in the combined sample; and
(e) from the concentration determined in (d), calculating the copy number of the target nucleic acid sequence in the query nucleic acid sample.

15. The method of claim 14, wherein the reaction volumes are droplets of an emulsion.

16. The method of claim 14, wherein the copy number of the first reference nucleic acid sequence in the diluting nucleic acid sample is known.

17. The method of claim 14, wherein the copy number of the second reference nucleic acid sequence in the diluting nucleic acid sample is known.

18. The method of claim 14, wherein the PCR reaction in (d) comprises pairs of primers that anneal to portions of the target nucleic acid sequence, the first reference nucleic acid sequence, and/or the second reference nucleic acid sequence.

19. The method of claim 14, wherein the calculation in (e) is performed by a computer.

20. The method of claim 14, wherein the query nucleic acid sample has at least 10-fold more copies of the target nucleic acid sequence than the first reference nucleic acid sequence.

21. The method of claim 14, wherein a quantity of the diluting nucleic acid sample is combined with a quantity of the query nucleic acid sample such that the ratio of the copy number of the target nucleic acid sequence to the copy number of the first reference nucleic acid sequence is between about 1 and 10.

22. The method of claim 14, wherein a quantity of the diluting nucleic acid sample is combined with a quantity of the query nucleic acid sample such that the copy number of the target nucleic acid sequence and the copy number of the first reference nucleic acid sequence are within about 20% of a minimal Poisson statistical uncertainty.

23. The method of claim 14, wherein the target nucleic acid sequence is a deoxyribonucleic acid sequence.

24. The method of claim 14, wherein the first and second reference nucleic acid sequences are deoxyribonucleic acid sequences.

25. The method of claim 14, wherein the second reference nucleic acid sequence is not found on the query nucleic acid sample.
